(19) United States
(12) Patent Application Publication
(10) Pub. No.: US 2006/0057165 A1
(43) Pub. Date: Mar. 16, 2006

(54) Clostridium botulinum toxin formulation and method for reducing weight

(76) Inventors: Dimitrios Dimitrakoudis, Toronto (CA); Helena Dimitrakoudis, Toronto (CA)

Correspondence Address: EDWARDS & ANGELL, LLP, P.O. BOX 55874, BOSTON, MA, 02205, US

(21) Appl. No.: 11/223,330

(22) Filed: Sep. 9, 2005

Related U.S. Application Data
(60) Provisional application No. 60/608,510, filed on Sep. 10, 2004.

(51) Int. Cl. A61K 39/08

(52) U.S. Cl. 424/239.1

(57) ABSTRACT

A method of altering taste sensation in an individual is provided, the method comprising administering an effective amount of botulinum toxin to the taste cells of the individual. The method can be used to effect reduced caloric consumption in an individual in need of reduced caloric consumption or to effect weight reduction in an individual in need of weight reduction.

### CROSS-REFERENCE TO RELATED APPLICATION

This application claims benefit and priority from U.S. provisional patent application No. 60/608,510, filed on Sep. 10, 2004, the contents of which are incorporated herein by reference.

### FIELD OF THE INVENTION

The present invention relates generally to methods of weight loss, and particularly to methods involving administration of a pharmaceutical compound.

### BACKGROUND OF THE INVENTION

Currently almost two thirds of adults (64%) in the United States are overweight, with obesity prevalence being approximately 30%. Obesity is a major risk factor for development of diabetes, heart disease, hypertension, obstructive sleep apnea, cancer and other disorders. Being overweight can also cause great anxiety and distress for its sufferers, leading to impairment in social function and affecting lifestyle choices. The economic impact of obesity in our population is tremendous, with an estimated total cost of about USD $130 billion per year in the United States alone.

Traditional therapies for weight loss include restrictive dieting and exercising. These almost always result in short term weight loss at best but not a long term sustainable solution. Many individuals will eventually relapse and may even gain more weight over time.

Other once promising approaches for reducing weight utilize drugs such as amphetamines and other related sympathomimetic medications, which stimulate the release of norepinephrine and/or dopamine from storage sites in nerve terminals in the lateral hypothalamic feeding center thus producing a decrease in appetite. These sympathomimetic medications have significant systemic cardiovascular effects, which can lead to serious arrhythmias and death, as in the case of Ephedra, the use of which has recently been cautioned against. Other adrenergic drugs for weight loss similar to amphetamine include phentermine. These drugs however have significant side effects such as insomnia, anxiety, dizziness, palpitations, headaches, intestinal cramping, diarrhea etc. Thus, since most of these weight loss drugs have significant side effects, use is often discontinued by the patient. Discontinuation often leads to a rebound in weight in the individual.

A newer weight loss medication is Meridia which acts centrally by increasing satiety. However, it has been linked with serious heart and cardiovascular complications. After 49 deaths, and many hundreds of serious complications, there is tremendous pressure to ban Meridia in North America, as has occurred in some European countries already.

Another newer weight loss medication is Xenical, which acts in the bowel by interfering with the absorption of fat. This medication has a modest effect in causing weight loss. Xenical also has very unpleasant and embarrassing side effects of excess flatulence, abdominal cramps, and foul smelling diarrhea, which limit its use.

Hormonal therapies for weight loss that are currently used include injections with growth hormones. However, this treatment is cost inhibitive and systemic use of growth hormones can have negative side effects on other systems of the human body, affecting the overall physiological homeostasis of the body.

More extreme methods of reducing weight include gastric surgery which works by altering the digestive processes. Restrictive operations reduce the size of the stomach, while malabsorptive operations work by bypassing parts of the intestine which absorbs most of the nutrients and calories. These procedures can be very invasive and costly, and carry a 15-20% risk of developing complications.

Liposuction is another weight loss treatment used in which fat deposits are surgically removed from the body. However, this method also carries many risks, including death during surgery if excessive fat is removed from the body.

The several above mentioned surgical procedures are often painful, invasive, time consuming, and costly, with significant drawbacks and often questionable benefits.

### SUMMARY OF THE INVENTION

In one aspect of the present invention there is provided a method for altering taste sensation in an individual and/or for reducing caloric intake in an individual and/or for reducing excess weight in an individual, comprising administering an effective amount of botulinum toxin to the taste cells of an individual in need of alteration of taste sensation and/or in need of reduced caloric intake and/or in need of weight reduction. In one embodiment, altering taste sensation comprises reducing taste sensation.

In another aspect of the present invention there is provided a formulation comprising botulinum toxin, adapted for administering the botulinum toxin to taste cells of an individual in need of alteration of taste sensation or to taste cells of an individual in need of reduced caloric intake or to taste cells of an individual in need of weight reduction.

In a further aspect of the present invention there is provided a kit comprising botulinum toxin and instructions for altering taste sensation in an individual in need of alteration of taste sensation or for reducing caloric intake in an individual in need of reduced caloric intake or for reducing excess weight in an individual in need of weight reduction.

Other aspects and features of the present invention will become apparent to those of ordinary skill in the art upon review of the following description of specific embodiments of the invention. The invention includes the following features hereinafter fully described in the detailed section of the preferred embodiment, but it should be noted that such a description discloses only some of the various ways in which the invention may be utilized.

### DETAILED DESCRIPTION

The inventors have developed a novel approach of using topical and injectable formulations of botulinum toxin as a method to reduce taste sensation, which can lead to decreasing caloric intake, thereby resulting in weight loss in an individual. Therefore, this approach may be used to treat excess weight and obesity, or as a cosmetic treatment for weight reduction, using minimal invasive procedures.

Taste modulation is one major potential mechanism for the treatment of excess weight and obesity. Previous research has shown that long term loss or suppression of taste sensation can lead to weight loss. A study by Poothullil has shown that subjects were able to terminate the act of eating when the pleasantness of flavor of food subsided during a meal. In this study, by the end of one month, significant weight loss took place in the study group and was maintained throughout the study period of one year (Maintenance of weight loss using taste and smell sensations (1999) J of Women's Health 8(1): 109-13). Earlier research has clearly shown that obesity and excess weight have to do with and are directly correlated with human eating behaviors, the palatability of food and the sensory specific satiety of food/perception of tastes (Nasser, J. Taste, food intake and obesity (2001) Obesity Reviews 2(4): 213-8).

Finsterer, et al. report about a particular case of loss of taste following a procedure to remove a vocal cord polyp (Loss of taste is loss of weight (2002) Lancet vol 359: 891). Gustatory tests revealed that taste was impaired on the tip of the tongue and taste perception was absent for sour, salt, bitter and reduced for sweet tastes. Immediately after the surgery the patient was slightly obese with a weight of 74 kg. During the next twenty months with taste impairment (ageusia) he lost 20 kg. The patient reported a loss of appetite as a result of loss of taste during eating and as a result, simply ate less. This published case underscores the potential benefits of taste modulation on excess weight and obesity.

Most species within the genus Clostridia produce bacterial toxins with a range of pathogenic effects such as food poisoning, tetanus and botulism. The virulent Clostridium botulinum strains are divided into seven groups and each respectively produces its own specific antigenically distinct toxin (Types A-G). The potency and the physiological modes of action of the above mentioned toxins vary but each normally leads to chemodenervation.

The neurotoxic component of botulinum toxin has a molecular weight of about 150 kDa and is thought to include a short polypeptide chain of about 50 kDa to which the neurotoxic properties of the toxin have been attributed.

Ingestion of C. botulinum toxin can in severe cases lead to botulism, a rare and sometimes fatal disease. The symptoms often include nausea, severe headaches, double vision, pharyngeal and limb weakness, paralysis, and autonomic failure. However, it is the same paralytic properties of C. botulinum toxin that have led to numerous medical uses of the toxin since it was produced in the mid-late 1900s. Various disorders and conditions treated by botulinum toxin include several neuromuscular disorders, dystonias, sympathetic neuronal disorders such as hyperhidrosis, inflammatory or pain disorders, cosmetic skin problems such as wrinkles, and neurological disorders such as headaches, tremors, etc.

The toxins produced by Clostridium botulinum species are proteins which interfere with release of neurotransmitters from nerve terminals by impairing vesicle translocation, docking and release at presynaptic membranes. Earlier research has shown botulinum toxins block the release of acetylcholine and other neurotransmitters from somatic nerve endings, preventing neuro-muscular transmission, and from autonomic nerves or peripheral nociceptive neuron terminals, leading to modulation of sweating and pain respectively.

In the present method, botulinum toxin is used to target the synapse between taste receptors and primary afferent neurons without affecting more deeply located motor nerves to the tongue. This also preserves tongue sensation of touch, temperature, and pain, as there is no superficial synapse involved in transmission of these sensations. The effect of the botulinum toxin applied at or near the tongue surface would be specific to taste sensation. The duration of effect from a single application would be at least two weeks, which is the turnover time for the taste receptors, but may be as long as three to twelve months, which is the range of duration of effect of botulinum toxin on muscle and autonomic nerves.

Thus, without being limited to any particular theory or mechanism of action, the present method relates to the ability of botulinum toxin to prevent synaptic transmission between taste cells and the adjacent neurons of the cranial nerves which lead to the brain. The loss of taste sensation in turn can lead to a significantly reduced appetite and a corresponding reduction in food or caloric intake, resulting in weight loss. In some instances, topical or injectable application of the botulinum toxin to the taste cells may lead to permanent loss of chemical synaptic transmission from affected taste cells. The affected cells would be permanently non-functional for the duration of their lifespan (at least two weeks for an individual cell). The clinical effect of such loss or alteration of taste perception and/or transmission would likely be longer than two weeks in certain individuals.

The precise neurotransmitters in taste cells have not yet been clearly delineated but several candidates have been identified. Based on the research to date, the following are potential candidates: acetylcholine, noradrenalin, serotonin, amino acids (glutamate and GABA) and peptides (substance P and CGRP).

In its broadest aspect, the invention relates to a method of altering taste sensation in an individual. Thus, in the present method, to alter taste sensation, botulinum toxin is administered to taste cells of an individual in need of altered taste sensation.

Altering taste sensation refers to interrupting, reducing or inhibiting an individual's ability to taste food, for example through an interruption of the signaling pathway involving taste cells and adjacent neurons, as well as to changing the way in which an individual perceives food flavours, such that food tastes different than it would have tasted without such altering. Thus, altered perception of taste may be achieved with or without an objective reduction in taste sensation. For example, altering may result in foods tasting unusually salty, bitter or metallic as a result of altering taste sensation in an individual, or foods may have generally less flavour as a result of altering taste sensation, or both. Altered perception of taste may lead to an alteration in the types of food, as well as the amount of food or calories, that the individual consumes.

Taste sensation refers to the ability to sense the flavour of food through an interaction of food flavour molecules with an individual's taste cells, for example through an interaction between flavour molecules and taste receptors located on the surface of taste cells.

An individual in need of altered taste sensation includes any individual for whom it is desired to have the ability to taste food altered from the level of taste that the individual experiences in the absence of treatment. This includes an individual in need of reduced caloric intake and/or in need of weight reduction, which includes an individual who is overweight, excessively overweight or obese, as well as an individual who desires to lose body weight for cosmetic reasons. Therefore, the term “excess weight” refers to an individual who is clinically overweight or obese, as well as an individual who may not be clinically overweight, but for whom it is desirous to reduce body weight. A skilled person will understand how to determine if an individual is clinically overweight or obese. An individual includes any animal, including a human, a dog or a cat.

As will be understood by a skilled person, taste cells are cells located primarily on the tongue and the soft palate and are involved in the perception of the taste of food. Taste cells express taste receptors and are ciliated neuroepithelial cells which form chemical synapses with primary neurites in taste buds superficially located on the tongue. The synapse between taste cells and the primary afferent neuron is also superficially located in the tongue, and hence are accessible to botulinum toxin when administered in accordance with the present inventions. Such cells emit neurotransmitters in response to an interaction of the taste receptors with flavour molecules in food, and stimulate adjacent neurons to transmit the taste perceptions to the brain. Flavour molecules are molecules within food that impart a taste or flavour to the food, and which are capable of stimulating the taste pathway through an interaction with taste cells.

The botulinum toxin may be any Clostridium botulinum toxin protein, including any of botulinum toxin types A-G, or may be a combination of different botulinum toxins, including one or more of botulinum toxin types A-G. Botulinum toxin can be obtained commercially, including from chemical and biological suppliers such as Wako (Osaka, Japan), Metabiologics (Madison, Wis.), and Sigma Chemicals (St Louis, Mo.), Dysport RTM (Ipsen), Myobloc (Solstice), and Allergan, supplied under the name BOTOX. A recombinantly produced botulinum toxin or derivative thereof may also be used. A recombinant botulinum toxin may be produced using standard molecular biology techniques known in the art, for example as described in Sambrook et al. ((2001) Molecular Cloning: a Laboratory Manual, 3^{rd} ed., Cold Spring Harbour Laboratory Press).

The term “botulinum toxin” includes homologs, fragments, derivatives or variants of botulinum toxin that possess the neurotoxic properties of botulinum toxin.

A polypeptide sequence is a “homolog” of, or is “homologous” to another polypeptide sequence if the two sequences have substantial identity over a specified region and the functional activity of the sequences is conserved (as used herein, the term “homologous” does not imply evolutionary relatedness). Two polypeptide sequences are considered to have substantial identity if, when optimally aligned (with gaps permitted), they share at least approximately 50% sequence identity, or if the sequences share defined functional motifs. In alternative embodiments, optimally aligned sequences may be considered to be substantially identical (i.e. to have substantial identity) if they share at least 60%, 70%, 75%, 80%, 85%, 90%, 95%, 96%, 97%, 98%, 99% identity over a specified region. An “unrelated” or “non-homologous” sequence shares less than 40% identity, and possibly less than approximately 25% identity, with a particular polypeptide over a specified region of homology. The terms “identity” and “identical” refer to sequence similarity between two peptides or proteins. Identity can be determined by comparing each position in the aligned sequences. A degree of identity between amino acid sequences is a function of the number of identical or matching amino acids at positions shared by the sequences, i.e. over a specified region. Optimal alignment of sequences for comparisons of identity may be conducted using a variety of algorithms, as are known in the art, including the ClustalW program, available at http://clustalw.genome.ad.jip, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis are available through the National Center for Biotechnology Information (through the internet at http://www.ncbi.nlm.nih.gov/). As used herein, “homologous amino acid sequence” includes any polypeptide having substantial identity to botulinum toxin, as described above, including polypeptides having one or more conservative substitutions, insertions or deletions, provided the polypeptide retains the neurotoxin function of botulinum toxin.

A variant or derivative of botulinum toxin refers to a botulinum toxin or a fragment thereof, which retains the neurotoxic properties of botulinum toxin, or a botulinum toxin that has been mutated at one or more amino acids, including point, insertion or deletion mutations, but still retains the neurotoxic properties of botulinum toxin. A variant or derivative therefore includes deletions, including truncations and fragments; insertions and additions, for example conservative substitutions, site-directed mutants and allelic variants; and modifications, including peptoids having one or more non-amino acyl groups (q.v., sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

The term “neurotoxic properties” or “neurotoxic function” of botulinum toxin refers to the ability of botulinum toxin to interfere with the release of vesicle-stored neurotransmitters into a synapse and to block, reduce or interfere with the interaction between taste cells and adjacent neurons, as well as to block, reduce or interfere with the synaptic transmission between neurons, and includes interference with or blocking of release of neurotransmitters involved in perception of taste, for example neurotransmitters such as acetylcholine, noradrenalin, serotonin, amino acids such as glutamate and GABA or peptides such as substance P and CGRP.

The botulinum toxin is administered so as to target the taste cells of the individual. Typically, botulinum toxin is administered locally, so as to prevent negative systemic effects of the toxin. Thus, in the present method, the botulinum toxin may be administered directly to the area containing the taste cells, including to the tongue or to the soft palate, using standard techniques known in the art, including topical administration, administration by injection, including by microinjection, and administration using a transdermal patch or a transmucosal patch, as discussed herein. In particular embodiments, the botulinum is applied to the taste cells, including on the tongue and/or soft palate, in the form of a topical cream or gel, by injecting a solution containing the botulinum toxin superficially into the tongue or soft palate with the aid of a plurality of microneedles on an injection plate, or with the aid of a transdermal or transmucosal delivery system or patch, or needleless injector device. Microinjections can be painful to the individual undergoing treatment and therefore, the tongue and/or soft palate could be anesthetized first with the aid of EMLA™ (Eutectic Mixture of Local Anesthetics) cream, an EMLA patch or a local nerve block injection.

An effective amount of botulinum toxin is administered to the taste cells of the individual. The term “effective amount” as used herein means an amount effective, at dosages, for periods of time and at intervals necessary to achieve the desired result, for example, to alter taste sensation in the individual, including altering taste sensation in the individual in a manner that effects a reduced caloric intake, potentially resulting in weight reduction in the individual.

The concentration and amount of the botulinum toxin to be administered will vary, depending on the desired purpose for reduction in taste sensation, the pharmacodynamic properties and type of botulinum toxin that is administered, the mode of administration, the age, sex, weight and health of the patient, the frequency of the treatment and the type of concurrent treatment, if any. A sufficient amount may be administered so as to result in altered taste sensation with a single treatment, but a skilled person will appreciate that the amount should not be so much as to permanently alter taste sensation in the individual. Although the neural structures acted on by the botulinum toxin are part of the peripheral nervous system rather than central nervous system and therefore capable of regenerating, a permanent loss of taste sensation due to too high of a dose would be extremely unlikely but possible in a patient with a disorder which impedes nerve regeneration such as extreme age or diabetes.

One of skill in the art can determine the appropriate amount of botulinum toxin for administration based on the above factors. The toxin may be administered initially in a suitable amount that may be adjusted as required, depending on the clinical response of the individual. The effective amount of toxin can be determined empirically and depends on the maximal amount of the toxin that can be administered safely, and the minimal amount of the toxin that produces the desired result. In some embodiments, the amount of botulinum toxin administered is less than the amount typically used for other current indications of the toxin.

In certain embodiments, the amount of botulinum toxin administered in a single treatment ranges from about 1 unit to about 20,000 units. In certain embodiments, from about 1 unit to about 20,000 units, from about 1 to about 15,000 units, from about 1 to about 10,000 units, from about 1 to about 5,000 units, from about 1 to about 1,000 units, from about 1 to about 500 units, from about 1 to about 200 units or from about 5 to about 100 units of botulinum toxin are administered to a patient in a single treatment. In different embodiments, about 1 unit, about 5 units, about 10 units, about 20 units, about 25 units, about 30 units, about 40 units, about 50 units, about 60 units, about 70 units, about 75 units, about 80 units, about 90 units, about 100 units, about 125 units, about 150 units, about 175 units, about 200 units, about 250 units, about 500 units, about 750 units, about 1000 units, about 1,500 units, about 2,000 units, about 2,500 units, about 3,000 units, about 5,000 units, about 7,500 units, about 10,000 units, about 15,000 units or about 20,000 units of botulinum toxin are administered to the individual in a single treatment. As used herein, one unit of botulinum toxin is the amount of botulinum toxin equivalent to the LD₅₀ in mice. The potency of botulinum toxin type A in humans as provided by Allergan under the registered trademark BOTOX is about LD₅₀=2,730 units (for parenteral administration).

The particular amount administered in a single treatment will vary depending on the particular formulation and mode of delivery. For example, a microneedle injection plate application would require much smaller amounts than topical applications since there is direct delivery to the subdermal structures without the barrier of the epithelium.

The effect of a single administration of botulinum toxin in altering taste sensation in an individual will vary. Typically, upon administration of an effective amount, the individual will notice the effect in altered taste sensation within 1 to 3 days following administration. The alteration in taste sensation may last about 2 weeks, from about 2 weeks to a about 1 year, or from about 2 weeks to about 6 months, or from about 2 weeks to about 3 months, or from about 2 weeks to about 2 months, or from about 2 weeks to about 1 month.

Compounds which are known to impair taste may also act synergistically with botulinum toxin. Thus, in certain embodiments, the botulinum toxin may be administered in combination with one or more other taste inhibitors. As used herein, a taste inhibitor is a substance that alters, inhibits, reduces or impairs taste sensation or perception. Such a substance, which is not botulinum toxin, may include, for example, an antimicrobial agent, an antifungal agent, an anti-inflammatory agent, an ACE inhibitor, a calcium channel blocker, an anti-arrhythmic agent, an anticholinergic agent, a selective serotonin reuptake inhibitor, a tricarboxylic acid medication, or an antipsychotic agent.

A combination of botulinum toxin and another taste inhibitor for administration may be formulated together in the same dosage form or may be formulated in separate dosage forms, and the separate dosage forms may be the same form or different forms, for administration by the same mode or by different modes of administration. Furthermore, administration of a combination of botulinum toxin and another taste inhibitor, when not together in the same dosage form, means that the botulinum toxin and the taste inhibitor are administered concurrently to the individual being treated, and may be administered at the same time or sequentially in any order or at different points in time. Thus, botulinum toxin and another taste inhibitor may be administered separately but sufficiently closely in time so as to provide the desired therapeutic effect.

Altering taste sensation using botulinum toxin as described in the above method may be used to effect reduced caloric intake in an individual in need of such reduced caloric intake and to treat excess weight and obesity in an individual in need of weight reduction. Sufficient alteration of taste sensation tends to lead to decreased caloric intake, which can lead to progressive, physiologic and safe loss of weight in many individuals. Thus, there is provided a method of reducing caloric intake in an individual in need of reduced caloric intake. There is also provided a method of reducing excess weight in an individual in need of weight reduction, in which an effective amount of botulinum toxin is administered to alter taste sensation in the individual, the alteration in taste sensation being effective for decreasing caloric consumption by the individual.

The botulinum toxin is administered as described above so as to alter taste sensation in the individual being treated for weight reduction. To effect weight loss, the administration of the botulinum toxin may be repeated as necessary to obtain the desired result. Administrations are typically given periodically, while monitoring any response. It will be recognized by a skilled person that lower or higher dosages than those indicated above may be given, according to the administration schedules and routes selected. That is, the botulinum toxin may be administered to the taste cells of an individual in repeated doses, the timing of the doses and the total period of time over which the doses are administered being adjusted for the individual based on factors including the individual's age, weight, sex, health, the duration of the effect of a single administration of the individual, the effect of a single administration on the individual's caloric consumption and the rate at which the individual loses weight as a result of reduced caloric intake. The dose, timing of repeated doses and the total time period over which repeated doses are given can be determined by a skilled person using standard clinical methods, for example while monitoring the effect on caloric intake and weight between applications of the drug.

In certain embodiments, the botulinum toxin is re-administered from about once every two months to once every year, from about once every three months to about once every year, from about once every four months to about once every year, from about once every five months to about once every year, from about once every six months to about every year, for the desired time period, or until the desired result is achieved, for example, a desired amount of weight reduction is achieved in the individual. In certain other embodiments, the botulinum toxin is re-administered about six months, about four months, about three months, about two months, following the previous administration. It should be noted that too frequent dosing may lead to antibody formation and loss of future efficacy, and thus an interval of at least 3 months between administrations is preferred.

To aid in administration, the botulinum toxin may be included in a formulation that is suitable for administering the botulinum toxin to taste cells of the individual, which may be targeted for example by administering botulinum toxin to the tongue or soft palate. Therefore, in a further aspect, there is provided a formulation comprising botulinum toxin, adapted for administering the botulinum toxin to taste cells of an individual in need of taste sensation reduction or in need of reduced caloric intake or in need of weight reduction. There is also provided in a further aspect, such formulations for use in reducing taste sensation in an individual in need of alteration of taste sensation or reducing caloric intake in an individual in need of reduced caloric intake or reducing excess weight in an individual in need of weight reduction.

A formulation that is adapted for administering the botulinum toxin to taste cells is a formulation that is suitable for topical, transdermal or injection administration to the tongue or soft palate. The formulations may routinely contain pharmaceutically acceptable concentrations of salt, buffering agents, preservatives and various compatible carriers. For all forms of delivery, the botulinum toxin may be formulated in a physiological salt solution, including in a physiological salt solution that is subsequently dried or lyophilised.

The formulation will typically include a pharmaceutically acceptable diluent or carrier. The proportion and identity of the pharmaceutically acceptable diluent is determined by chosen route of administration, compatibility with the botulinum toxin protein and any other pharmacologically active ingredient that may be included in the formulation, and standard pharmaceutical practice. Generally, the formulation will include components that will not significantly impair the neurotoxic properties of the botulinum toxin, or cause degradation of or reduce the stability the toxin.

The toxins produced by Clostridium botulinum are relatively large and drug penetration may be hampered by low permeability of the tongue because of the barrier properties of the skin and saliva which allow for the passage of small polar molecules such as water and oxygen, but which can block the penetration of larger molecules. Thus, the formulation of botulinum toxin may include a transdermal delivery enhancer which does not affect the bioactivity of the toxin to enhance permeability and penetration of the botulinum toxin when the formulation is applied to the tongue or soft palate, such as a chemical enhancer. The chemical enhancer may be, for example, a surfactant, a lipid, an aliphatic compound, a liposome, a noisome, a hyaluranidase or combinations thereof.

Alternatively, other enhancers may be used with a particular formulation to increase delivery of the botulinum toxin to the taste receptors, including mechanical transdermal delivery enhancers for example tongue surface cleaners or scrapers, electrical transdermal enhancers for example electroporation devices, or biological transdermal delivery enhancers for example, enhancers lipophilic vesicles.

The formulation may be formulated as a water based formulation, including diluted with or provided in saline, or it may be formulated as a gel, an ointment, a cream, an emulsion, a microemulsion, a temporary adhesive patch, or as a solution for use with a needleless injector device or a multineedle injection plate. Microemulsion formulations of botulinum toxin can be utilized to improve absorption coefficients when compared with the traditional approaches to developing topical formulations.

In certain embodiments, the formulation is a gel or a cream suitable for topical administration to the tongue or soft palate, a transdermal adhesive patch for application to the tongue or soft palate, or a solution suitable for injection into the tongue or soft palate.

In one embodiment, the formulation is a topical gel containing the botulinum toxin that can be applied topically to the taste cells, for example to the area of the tongue or soft palate. Preparation of topical gels are generally known in the art. For example, the gel may contain an appropriate amount of the toxin for delivering a single dose in one application, and may also contain an enhancer as described above to increase delivery of the toxin to the taste receptors, as well as other excipients and components required to form the gel, for example a gelling agent.

Similarly, in a different embodiment, the formulation is a topical cream containing an appropriate amount of botulinum toxin for topical application. Preparation of topical creams are also known in the art. The cream contains an appropriate amount of the toxin, and may also include an enhancer, a hydrophobic component and a hydrophilic component as well as a surfactant so that the cream can be formulated as a microemulsion to increase the delivery of the toxin to the taste receptors, as well as any necessary diluents, carriers or excipients.

In a different embodiment, the formulation is a transdermal or a transmucosal patch comprising botulinum toxin. For example, the toxin may be supplied in a plurality of microwells or pockets sealed by a dissolvable membrane on the mucosal side of a transdermal adhesive patch. The membrane may contain cellulose or starch, and may further contain one or more compounds to aid in permeation of the botulinum toxin into cells, for example polyvinyl alcohol, polyethylene oxide, or hydroxypropyl methyl cellulose. The botulinum toxin may be stored in a dried or lyophilized state and may be solubilized by wetting the patch with saline. Alternatively, the patch may contain one or more pockets of solubilizing solution (saline and enhancing agent) that will rupture with pressure causing the fluid to mix with the neurotoxin. The patch may be applied to the tongue or soft palate by placing it on a mucoadhesive matrix layer, or it may have the adhesive attached to the mucosal surface of the patch.

In a different embodiment of the formulation that is also a transdermal or transmucosal patch formulation, the toxin is included in a mixture that may also contain a stabilizer and an enhancer. This mixture is then incorporated into the adhesive layer of the transdermal or transmucosal adhesive patch.

Various mucoadhesives can be used within a gel or cream formulation or on a transdermal adhesive patch to establish adhesive contact with mucosal surfaces. The following patents disclose examples of mucoadhesives: U.S. Pat. No. 5,700,478; U.S. Pat. No. 4,259,314; U.S. Pat. No. 4,680,323; U.S. Pat. No. 4,740,365; U.S. Pat. No. 4,573,996; U.S. Pat. No. 4,292,299; U.S. Pat. No. 4,715,369; U.S. Pat. No. 4,876,092; U.S. Pat. No. 4,855,142; U.S. Pat. No. 4,250,163; U.S. Pat. No. 4,226,848; U.S. Pat. No. 4,948,580; and U.S. Reissue Pat. Re. 33093, all of which are fully incorporated herein by reference. Such mucoadhesives are also described in J. Robinson, 18 Proc. Intern. Symp. Control. Rel. Bioact. Mater. 75 (1991), which is fully incorporated herein by reference. These adhesives usually consist of a matrix of a hydrophilic, eg. water soluble or swellable polymer or mixture of polymers, which can adhere to wet mucosal surfaces. Such polymers include hydoxypropyl cellulose, hydroxypropyl methylcellulose, hydroxy ethylcellulose, ethylcellulose, carboxymethylcellulose, dextran, guar-gum, polyvinyl pyrrolidone, pectins, starches, gelatin, casein, acrylic acid, acrylic acid esters, acrylic acid copolymers, vinyl polymers, vinyl copolymers, vinyl alcohols, alkoxy polymers, polyethylene oxide polymers, polyethers and the like. The mucoadhesive may also contain additives to enhance the local delivery of the botulinum toxin.

Various transdermal or transmucosal delivery systems have been developed, including those described in U.S. Pat. No. 5,516,523; U.S. Pat. No. 6,210,699; U.S. Pat. No. 6,488,953; U.S. Pat. No. 6,585,997, all of which may be adapted for use in the presently provided formulation, and all of which are herein fully incorporated by reference.

The transdermal or transmucosal patch may also contain a plurality of microneedles, solid or hollow. The needles would be of an appropriate length such that they would contact the taste cells, but would not penetrate blood vessels or any muscle, for example in the tongue or soft palate.

Similarly, the formulation may include an appropriate amount of botulinum toxin formulated into a solution for injection into the superficial layer of the tongue or soft palate containing the taste cells, for example using a plate injector device with a plurality of microneedles. For example, the toxin may be dissolved in a saline solution, buffered to a physiological pH, and optionally also including an enhancer, which has a viscosity that allows for easy syringability using the plate injector device. The various formulations suitable for injectable use include sterile aqueous solutions or dispersions, as well as sterile powders for the extemporaneous preparation of sterile injectable solutions or dispersions. In all cases the form must be sterile and must be fluid to the extent that easy syringability exists.

As mentioned above, the botulinum toxin may be administered in combination with other taste inhibitors. Thus, the formulation may further include a taste inhibitor in addition to the botulinum toxin, for example without limitation, one or more of an antimicrobial agent, an antifungal agent, an anti-inflammatory agent, an ACE inhibitor, a calcium channel blocker, an anti-arrhythmic agent, an anticholinergic agent, a selective serotonin reuptake inhibitor, a tricarboxylic acid medication, or an antipsychotic agent.

The formulation can be prepared by known methods for the preparation of pharmaceutically acceptable formulations suitable for administration to patients, such that an effective quantity of the active substance or substances is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

The formulation is generally prepared in order to deliver an appropriate amount of botulinum toxin in a single dose of the formulation. The determination of an appropriate amount of botulinum toxin is described above, and depends on the particular condition being treated, the severity of the condition, the individual patient parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation.

The botulinum toxin, or a formulation comprising the botulinum toxin may also be packaged as a kit or commercial package, containing instructions for use of botulinum toxin, including the use of botulinum toxin to reduce taste sensation in an individual in need of taste sensation reduction, use of botulinum toxin to reduce caloric intake in an individual, or use of botulinum toxin to reduce excess weight in an individual in need of weight reduction.

The present invention also contemplates the use of botulinum toxin to alter taste sensation in an individual in need of taste sensation alteration. There is also provided use of botulinum toxin to reduce caloric intake in an individual in need of reduced caloric intake. There is further provided use of botulinum toxin to reduce excess weight in an individual in need of weight reduction. In one embodiment the individual is clinically overweight or obese. In another embodiment the individual desires to lose excess weight for cosmetic reasons. There is also provided use of botulinum toxin in the manufacture of a medicament for altering taste sensation in an individual in need of taste sensation reduction, for reducing caloric intake in an individual or for reducing excess weight in an individual in need of weight reduction.

The following examples describe formulations and methods encompassed by the present invention to treat patients, and are not intended to limit the scope of the invention.

### EXAMPLES

For each of the below examples of formulation of botulinum toxin, the formulation may be placed on the tongue or the soft palate of the individual to be treated. Typically, the formulation may be contacted with the tongue or soft palate for, for example, up to 30 minutes. Up to 3 days following treatment, the individual may experience a loss or alteration of taste sensation which may last from 2 weeks to 3 months, depending on the individual.

During the application period, a suction device and absorbent gauze (similar to that used in a dental office) can be inserted into the patient's mouth to remove saliva prior to application, and to keep the tongue and soft palate area dry throughout the application process.

Generally, certain chemical agents might be used within formulations of the toxin to enhance penetration when applied to the tongue. Such agents might include surfactants, lipids, liposomes and other aliphatic compounds. These agents can be used to improve toxin permeability. Microemulsion formulations of topical agents can also be utilized to improve absorption coefficients when developing topical formulations. Hyaluranidase has also been shown to help drug delivery of botulinum toxin from previous research and studies and may also be used in the present formulations to enhance drug delivery and permeability.

### Example 1

### Topical Formulation of Botulinum Toxin for Application to Tongue or Soft Palate

100 units or less of botulinum toxin type A and/or B (or other types and/or combinations of toxins) is suspended in a cream vehicle consisting of water, mineral oil, glycerin, cetyl alcohol, propylene glycol, methyl paraben and methyl cellulose. Alternatively, the toxins are suspended into a gel formulation composed of alcohol, water, propylene, and hydroxypropylcellulose. The dosage is varied depending on the age, weight, sex of the individual.

The above formulations are suitable vehicles for a single dose of up to 100 units of botulinum toxin being applied to the tongue. The formulation remains in contact with the superior surface of the tongue and/or soft palate, for example, covered by a fitted, non-permeable plastic covering, for a specified amount of time, for example, between 1-30 minutes. The topical formulation is wiped off after the application time is completed, and the mouth rinsed.

The following sample formulations illustrate exemplary topical formulations which may be applied topically on the tongue or soft palate for use in the present methods, as described in U.S. patent application 20030113349, which is herein incorporated by reference.

TABLE 1
Sample gel formulation:

| | |
|---|---|
| Botulinum toxin B | 50 units |
| Hydroxypropylcellulose | 0.50% |
| Preservative | 0.15% |
| Solvent/Ethanol | 7.5% |
| Antioxidant | 0.025% |
| Water | qs 50% |
| Klucel H gelling agent (by Hercules) | |

TABLE 2
Sample cream formulation (oil in water emulsion):

| | |
|---|---|
| Botulinum toxin G | 100 units |
| Glyceryl mono-distearate | 2.00% |
| Cetyl alcohol | 1.50% |
| Cetylstearyl alcohol | 7.00% |
| Polydimethylsiloxane | 1.50% |
| Liquid petroleum jelly | 17.50% |
| Preservative | 0.30% |
| Fragrance/non allergenic | 0.50% |
| Glycerol | 12.50% |
| Water | qs 100% |

### Example 2

### Transdermal Patch Formulation of Botulinum Toxin for Application to the Tongue or Soft Palate

Lyophilized botulinum toxin is contained within a series of wells located on the dermal side of a transdermal patch. The patch is shaped in the average size and shape of a tongue. The wells are organized in grids (0.5 cm×0.5 cm) and each grid contains an average of 50-100 wells. The dermal side of the patch is made of polyethylene terephthalate (PET). Each well contains 10-100 units of lyophilized botulinum toxin. The wells are sealed with a dissolvable membrane film made of polyvinyl alcohol, polyethylene oxide, and hydroxypropyl methyl cellulose. A rubber adhesive border is applied around the grid (e.g. using an adhesive such as R1072 from B.F. Goodrich Co.). U.S. patent application 20040009180 is referenced, and is herein incorporated by reference.

This patch may be stored safely at 4 degrees Celsius for a few months.

The tongue and/or soft palate of the patient is air dried, and the patch applied for a specified amount of time (for example, 5-30 minutes).

### Example 3

### Transdermal Patch Formulation for Application of Botulinum Toxin to the Tongue or Soft Palate

A transdermal patch contains a depot of dried botulinum toxin, and a pocket of saline. The patch is applied to the tongue and/or soft palate and pressure is applied to rupture the saline pouch so that it mixes and dissolves the toxin. The formulation then diffuses through the areas covered by the patch. The adhesive prevents the solution from leaking outside of the patch area.

The patch is left in place for 5-30 minutes while suctioning the patient's mouth.

All documents referred to herein are fully incorporated by reference.

All technical and scientific terms used herein have the same meaning as commonly understood by one of ordinary skill in the art of this invention, unless defined otherwise.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. The invention is intended to encompass all such modification within its scope, as defined by the claims.

What is claimed is:

1. A method for altering taste sensation in an individual and/or for reducing caloric intake in an individual and/or for reducing excess weight in an individual, comprising administering an effective amount of botulinum toxin to the taste cells of an individual in need of alteration of taste sensation and/or in need of reduced caloric intake and/or in need of weight reduction.

2. The method of claim 1 wherein altering comprises reducing.

3. The method of claim 1 wherein the individual is clinically overweight or obese.

4. The method of claim 1 wherein the botulinum toxin is botulinum toxin type A, B, C, D, E, F or G, or a combination thereof.

5. The method of claim 1 wherein administering to the taste cells comprises administering to the tongue or the soft palate of the individual.

6. The method of claim 5 wherein administering comprises applying a topical formulation to the tongue or soft palate of the individual.

7. The method of claim 5 wherein administering comprises microinjecting into the tongue or soft palate of the individual.

8. The method of claim 5 wherein administering comprises applying a transdermal or transmucosal patch to the tongue or soft palate of the individual.

9. The method of claim 1 wherein between about 1 unit and about 20,000 units of botulinum toxin are administered.

10. The method of claim 1 wherein the amount of botulinum toxin administered is effective for altering taste sensation for up to about 6 months.

11. The method of claim 1, further comprising administering to the individual a taste inhibitor other than botulinum toxin.

12. The method of claim 11 wherein the taste inhibitor other than botulinum toxin comprises an antimicrobial agent, an antifungal agent, an anti-inflammatory agent, an ACE inhibitor, a calcium channel blocker, an anti-arrhythmic agent, an anticholinergic agent, a selective serotonin reuptake inhibitor, a tricarboxylic acid medication, or an antipsychotic agent.

13. The method of claim 1 further comprising re-administering the effective amount of botulinum toxin.

14. The method of claim 13 wherein the re-administering occurs about 6 months subsequent to said administering.

15. The method of claim 13 wherein said re-administering occurs from about two months to about 6 months subsequent to said administering.

16. A formulation comprising botulinum toxin, adapted for administering the botulinum toxin to taste cells of an individual in need of alteration of taste sensation or to taste cells of an individual in need of reduced caloric intake or to taste cells of an individual in need of weight reduction.

17. The formulation of claim 16 wherein the formulation is a topical gel or a topical cream.

18. The formulation of claim 16 wherein the formulation is a solution for microinjection.

19. The formulation of claim 16 wherein the formulation is a transdermal or transmucosal patch.

20. The formulation of claim 19 further comprising a mucoadhesive.

21. The formulation of claim 16 wherein the botulinum toxin is botulinum toxin type A, B, C, D, E, F or G, or a combination thereof.

22. The formulation of claim 16 further comprising a chemical enhancer.

23. The formulation of claim 22 wherein the chemical enhancer comprises a surfactant, a lipid, an aliphatic compound, a liposome, a noisome or a hyaluranidase, or combinations thereof.

24. The formulation of claim 16 further comprising a taste inhibitor other than botulinum toxin.

25. The formulation of claim 24 wherein the taste inhibitor other than botulinum toxin comprises an antimicrobial agent, an antifungal agent, an anti-inflammatory agent, an ACE inhibitor, a calcium channel blocker, an anti-arrhythmic agent, an anticholinergic agent, a selective serotonin reuptake inhibitor, a tricarboxylic acid medication, or an antipsychotic agent.

26. A kit comprising botulinum toxin and instructions for altering taste sensation in an individual in need of alteration of taste sensation or for reducing caloric intake in an individual in need of reduced caloric intake or for reducing excess weight in an individual in need of weight reduction.

27. The kit of claim 26 wherein the botulinum toxin is formulated in a formulation adapted for administering the botulinum toxin to taste cells of an individual in need of alteration of taste sensation or to taste cells of an individual in need of reduced caloric intake or to taste cells of an individual in need of weight reduction.

28. The kit of claim 27 wherein the formulation comprises a topical gel or a topical cream.

29. The kit of claim 27 wherein the formulation comprises a solution for microinjection.

30. The kit of claim 27 wherein the formulation comprises a transdermal or transmucosal patch.
